(19) United States
(12) Patent Application Publication
(10) Pub. No.: US 2013/0164738 A1
(43) Pub. Date: Jun. 27, 2013

(54) Genetic Sample Collection Systems

(71) Applicants: David Becker, San Diego, CA (US); James Plante, Del Mar, CA (US); Moreno Tanya, San Diego, CA (US); Cindy Wang, San Diego, CA (US)

(72) Inventors: David Becker, San Diego, CA (US); James Plante, Del Mar, CA (US); Moreno Tanya, San Diego, CA (US); Cindy Wang, San Diego, CA (US)

(73) Assignee: Pathway Genomics

(21) Appl. No.: 13/333,883

(22) Filed: Dec. 21, 2011

(51) Int. Cl. C12Q 1/68; C12M 1/00

(52) U.S. Cl. 435/6.1; 435/306.1

(57) ABSTRACT

Biological sample collection kits are devised with physical features to enable a high performance collection system which delivers preprocessed biological matter via conventional shipping means to a testing laboratories. In particular, untrained and unskilled users deposit biological matter such as saliva or blood into a receiving vessel. By sealing the container, the user causes release of a premixed solution containing preservatives and optionally lysis reagents. In addition, a purification agent is arranged to bind to target molecules and facilitate their removal from solution. These time consuming processes occur while the sample is in transit to the testing facility such that when it arrives, it is in a preconditioned state immediately ready for execution of washing steps. Thus, the high performance containers taught herein are useful for collection biological samples and performing initial process steps on received matter—steps which are largely effected during the shipping stage of the transfer process.

### BACKGROUND OF THE INVENTION

1. Field

The following invention disclosure is generally concerned with apparatus and technique for the collection of genetic samples and specifically concerned with systems which include DNA stabilization and purification functionality incorporated therein.

2. Prior Art

Container arts and engineering is at an advanced state where container designs and function are configured in support of particular purposes. For example, containers are sometimes molded into complex shapes from plastic materials in support of anti-theft features. The container protects an enclosed product as the plastic from which it is made is highly resistant to tearing and opening and portions thereof are held together with durable plastic welds while the package further includes devices such as RFID tags or other radio transmitter systems in communication with anti-theft means.

Containers and/or product packages designed for other applications also are highly advanced with many incorporated features designed to cooperate with the nature of the service for which it is designed. This is particularly true for containers designed to collect biological material and even more particularly when those containers are to be used without assistance from medical professional experts—i.e. for use by untrained consumers in a setting where professionals may not be present. Further, such containers must sometimes support special features for long-term storage, durability, chemical processing, safe handling, transmission via postal and delivery services, among others.

In some special cases, it is desirable to provide a container suitable for safe transmission of biological matter via the mails and/or private delivery services. For example, packages may be arranged with a view towards use with private delivery systems and services such as those provided by Federal Express. In this regard, a package system must be provided to protect against shock, large temperature changes, time delay, et cetera—those conditions that might be associated with the normal procedures used by delivery service companies. It is further desirable that containers are designed for ease of use by non experts without need for special training—and without complex instructions and procedures. In certain applications, containers for collection of biological samples, biomaterials, et cetera, are to be used by a donor without assistance of experts or medical professional. In home collection systems, the container must be highly functional without complex instructions and further it must be without failure mechanisms which can render collection of a sample defective. Thus, containers which effectively capture a sample with a minimal amount of steps and a minimal number of parts is a great benefit with respect to system success.

A number of systems have been developed for handling viscous liquids, particularly saliva and blood serum. See, for example, Haldopoulos, U.S. Pat. No. 3,832,141; Ohringer, U.S. Pat. No. 3,846,077; Breno, U.S. Pat. No. 4,209,488; Mar, U.S. Pat. No. 4,644,807; Romer, U.S. Pat. No. 4,895,808; and Seymore, U.S. Pat. No. 5,268,148. However, those apparatus that have previously been developed in this field are generally sophisticated devices intended for use by a skilled laboratory technician.

D'Angelo teaches in U.S. Pat. No. 7,387,999 dated Jun. 17, 2008 a saliva sample collection system. A sponge portion is used to swap saliva and saliva is thereafter extracted from the sponge by squeezing or centrifuge.

In a disclosure entitled “Apparatus for Sampling, Storing, Preserving and Testing a Specimen”, inventor Alley of Pennsylvania presents a swab tip and cooperating container system which is arranged to compress the swap when inserted therein. A portion of the sample is conveyed into a plurality of separate chambers provided to isolate each from the others. As the kit is designed around multipurpose functions related to testing directly within the device, the complexity is significant. In applications where only a single sample is required, or systems where testing is done wholly external to the sample collection kit, this system does not provide advantage.

In U.S. Pat. No. 5,981,293, a full description for a fluid collection kit and method is published. Biex Inc, of California as assignee uses a fluid collection, filtration and storage device in connection with biological matters such as saliva, among others. In particular, the device has a first tube with a closed first end and open second end, and a second concentric tube acting in concert therewith. Also, the system deploys a cap to form a liquid tight seal at the outer tube orifice.

Inventors Chess et al, present their invention in U.S. Pat. No. 5,674,456 of Oct. 7, 1997. A container with specific design directed to a ‘transportable container’ for a medical specimen—takes the design of a ‘Coplin’ jar. “A lid hingedly coupled to the top of the jar” accommodates both ‘open’ and ‘closed’ positions. Included in these systems is a tray which receives the container in a “sideways fashion”. However, this tray does not support a dual-mode accommodation for the jar.

Minnesota inventors Lenmark Sr. and Koentopp teach a specialized kit particularly suited for shipping transport. The container provides for a foam member with prescribed cut-outs to receive sample containing vials therein. While these cut-outs are not designed in support of any dual-mode shipping objectives, they do indeed accompany a plurality of elements in an application specific shipping box.

One kit manufactured by DNA Genotech Co. of Ontario, Canada is quite widely used by many professionals of the DNA diagnostics industries. However these systems have many complexities and shortcomings which contribute to failure mechanisms resulting in a unreliable system. A first important shortcoming of the Genotech system is that it is comprised of four discrete parts which must repeatedly be coupled and decoupled in a series of complex steps which must be executed in a particular order. While the kit includes a detailed instruction booklet in six languages with a series of grayscale photo-like diagrams, the steps illustrated are difficult to execute without error for some users.

In one important example, a receiving vessel is coupled by way of a thread set to a funnel element. The funnel is provided so that a user can easily spit into the device and the received saliva will be conveyed into the small aperture of the receiving vessel. After a sufficient quantity of saliva is received into the vessel, the funnel must be decoupled from the receiving vessel by twisting the funnel about an axis in a rotational direction opposite to that of which the vessel is twisted. However, this must be done only after a special DNA preserving and stabilizing fluid is introduced to the receiving vessel. To effect this, another thread set system is coupled to the funnel at an opposing end. That is, at the large end of the funnel, a specifically prepared container of fluid is screwed onto the funnel at its top. As the container is applied in this way, a membrane is pierced and fluid released into the funnel through which it passes and finally received into the receiving vessel to mix with the saliva. Then, the funnel may be finally decoupled from the receiving vessel. Thereafter, a stopper cap with a thread set identical to that of the funnel's narrow end is screwed onto the vessel aperture end to form a seal thereby trapping and containing both collected saliva and stabilization fluid therein.

The funnel and large cap/container are left as waste material. The reservoir contains residue chemicals which tend to cause anxiety in some persons. While it is not necessary to discard these pieces as medical waste, these leftover parts tend to at least have the appearance of medical waste and thus give rise to worry and a need for special and sometimes expensive treatment. It is surely preferable to devise a system with no leftover parts to be discarder as medical waste. Because the funnel needs to be coupled to and decoupled from the receiving vessel, it is formed with integrated finger grips which are easily engaged and ergonomically cooperative with human finger tips. However if the requirement that the funnel be screwed ‘on’ and ‘off’ the receiving vessel were removed, then so would the need for these finger grips; thus simplifying manufacturing processes and saving material. Another important problem with the collection systems described relates to spilling. Because a considerable amount of saliva is required to properly fill the receiving vessel, it generally takes a user some time to provide this quantity. During the process of filling the container, one may wish to set it down while time passes for additional saliva to form in the mouth. However, due to another design shortcoming, the device must continuously be held and cannot easily be put down without spilling its contents. It would be yet another important improvement if the container could rest on its own structure to allow a user time to fill the apparatus without having to continuously hold the device.

Systems provided for use with common postal mailing services require a stabilization fluid to preserve the sample for an extended period of time. Once biomaterial is received in the container, that material is mixed with a preserving fluid whereby the material is protected from natural degradation. However, no further chemical processing is anticipated. Once the collected material arrives at the laboratory, it may then be processed in a series of steps to further advance and prepare that material for genetic testing. However, such serial application of process steps tends to be time consuming to the disadvantage of the process. As the shipping step takes a considerable amount of time, it would be highly desirable if this time could also be used to effect other time consuming steps of the entire process. Thus where a container is arranged to support simultaneous shipping and other chemical processing, the overall system efficiency is improved. As such, a high performance sample collection system would include means to bring about several of the process steps which can be completed during the shipping period.

While systems and inventions of the art are designed to achieve particular goals and objectives, some of those being no less than remarkable, these inventions of the art have nevertheless include limitations which prevent uses in new ways now possible. Inventions of the art are not used and cannot be used to realize advantages and objectives of the teachings presented here following.

### SUMMARY OF THE INVENTION

Comes now, David Becker, James Plante, Tanya Moreno, and Cindy Wang with an invention of genetic sample collection systems including devices and methods arranged to receive, preserver, purify and convey samples to a testing facility. It is a primary function of these sample collection systems to provide easy-to-use devices for collection of DNA samples. It is a contrast to prior art methods and devices that systems first presented here include integrated therewith means for processing and conditioning collected matter with a view to and in support of genetic testing. A fundamental difference between systems of the instant invention and those of the art can be found when considering their ability to manipulate received biological matter to put it in a preferred state that supports downstream DNA testing processes.

These systems include devices and methods particularly suited and configured for ease-of-use by unskilled and untrained users. These systems are further configured for safe conveyance of collected biomaterial via standard shipping services from a user/patient to qualified processing facilities. It is a primary function of these collection systems to provide easy-to-use biomaterial sample collection kits for direct-to-consumer uses. It is a contrast and distinction with respect to the state of the art that the systems first taught and presented herein additionally support lysing and purification functionality.

An apparatus for biomaterial sample collection, temporary storage, stabilization, lysing, purification and conveyance via common carrier delivery services is formed of two cooperating primary elements—each of these two elements having a plurality of features integrated therewith. Preferably formed of plastics molding processes, these two elements cooperate together to operate in ‘coupled’ and ‘decoupled’ states. A receiving vessel and sealing cap which couple together in best versions via mechanical interlock, in example a threaded type coupling, form a liquid tight containment cavity suitable for long-term and durable storage of biomaterials, for example human saliva samples containing DNA.

Important features are provided to support collection, storage, stabilization, lysing, and purification of DNA. These features are integrated with the device receiving vessel and sealing cap. Particularly, a receiving vessel may be provided include an integrated funnel portion which allows ergonomic cooperation with the human mouth with regard to spitting and efficiently receives saliva from a donor as the donor spits into a large aperture of the funnel at its top end. The funnel terminates at a narrow, generally cylindrical tubular portion suitable for containing liquids received therein.

In some important versions, a tubular portion may additionally include a specially prepared inside surface arranged to support advanced function. This surface may be prepared with specific molecules which operate to capture and hold DNA molecules. DNA binding molecules affixed to the interior surface of receiving vessel may be used in a purification process where DNA released in a as part of a lysate is separated from other cellular matter.

A receiving vessel additionally has integrated therewith an outer body portion which forms a rigid standing means upon which the system may rest upright on flat surfaces. This outer body portion may support additional structure such as a viewing window and functional indicia. A receiving vessel may also include an integrated knife element arranged to interact with portions of the sealing cap.

A sealing cap has integrated therewith a seal mechanism, piercable liquid reservoir, grip surface, label receiving surface, among others. When a sealing cap element is coupled to a receiving vessel by way of a mechanical interlock such as screw threads, the action of forming a seal between the cap and vessel simultaneously causes breach to the integrity of an integrated liquid reservoir such that the reservoir contents are released into and mix with biomaterial matter already in the receiving vessel.

High performance sample collection containers described herein are also arranged as a kit. A kit is specifically designed with system objectives in mind. Specifically, a primary purpose of the system is to support conveyance of a biological sample from an unskilled user to a testing facility. Accordingly, these kits include shipping container and supporting effects to further simplify use of the devices. An application-specific shipping container provides two operational modes for shipping and delivery of these devices. That is, the shipping container supports containment of a receiving vessel and sealing cap held separate from each other in a ‘decoupled’ state, and additionally provides for containment of a receiving vessel and sealing cap in a ‘coupled’ state. In a shipping mode that supports the apparatus characterized as in a ‘decoupled’ state, the device is shipped to a user/customer. In a shipping mode that supports the apparatus characterized as in a ‘coupled’ state, the system is return shipped to a laboratory or testing facility for additional processing and analysis. Thus this invention additionally contemplates cooperative shipping facility and kits arranged as special-purpose shipping containers designed to further support the tasks and objectives particular to direct-to-consumer saliva sample collection.

### OBJECTIVES OF THE INVENTION

It is a primary object of the invention to provide biological matter collection apparatus and methods.

It is an object of the invention to provide easy-to-use means for receiving, preserving, purifying and conveying genetic samples.

It is a further object to provide sample collection apparatus with integrated processing facility for conditioning samples for genetic testing.

A better understanding can be had with reference to detailed description of preferred embodiments and with reference to appended drawings. Embodiments presented are particular ways to realize the invention and are not inclusive of all ways possible. Therefore, there may exist embodiments that do not deviate from the spirit and scope of this disclosure as set forth by appended claims, but do not appear here as specific examples. It will be appreciated that a great plurality of alternative versions are possible.

### BRIEF DESCRIPTION OF THE DRAWING FIGURES

These and other features, aspects, and advantages of the present inventions will become better understood with regard to the following description, appended claims and drawings where:

FIG. 1 is a cross section diagram of one version of a sample collection system of the invention;

FIG. 2 illustrates one version of a mechanical interlock coupling between a cap and a receiving vessel;

FIG. 3 shows action of a fluid releasing into a collected sample; and

FIG. 4 shows a collection kit system which further supports objectives of sample collection by untrained users.

### GLOSSARY OF SPECIAL TERMS

Throughout this disclosure, reference is made to some terms which may or may not be exactly defined in popular dictionaries as they are defined here. To provide a more precise disclosure, the following term definitions are presented with a view to clarity so that the true breadth and scope may be more readily appreciated. Although every attempt is made to be precise and thorough, it is a necessary condition that not all meanings associated with each term can be completely set forth. Accordingly, each term is intended to also include its common meaning which may be derived from general usage within the pertinent arts or by dictionary meaning. Where the presented definition is in conflict with a dictionary or arts definition, one must consider context of use and provide liberal discretion to arrive at an intended meaning. One will be well advised to error on the side of attaching broader meanings to terms used in order to fully appreciate the entire depth of the teaching and to understand all intended variations.

### Purification System

A purification system of this disclosure includes systems which operate to separate DNA from a solution. A purification system includes a transfer vehicle that is arranged to capture and hold DNA locally whereby a washing step may be used to remove and separate unwanted matter from DNA.

### Transfer Vehicle

A transfer vehicle may be arranged as a great plurality of particle or bead elements or alternatively as a large surface area. In both cases, a ‘transfer vehicle’ includes a surface having thereon DNA binding molecules, the surface being operable for capturing and binding DNA thereto the transfer vehicle.

### Beads and/or Particles

Beads or particles may be mixed with fluid solutions and may include at their surface binding molecules which attach to DNA free-floating in solutions.

### Shipping Services

Public mails and postal services as well as private express delivery services for example Federal Express and United Parcel Service comprise those shipping services and similar terms used herein.

### PREFERRED EMBODIMENTS OF THE INVENTION

Containers for the collection and conveyance of biomaterials, and specifically saliva with human DNA therein, are arranged in an easy-to-use manner for use by unskilled consumers. These container systems are provided with advanced functionality which is largely automatic, built-in and integrated such that collected saliva is subject to pre-processing steps during transit to test facilities. Users of these devices and kits do not have to perform any laboratory procedures or complex chemical processing but rather they simple spit into a receiving vial and seal with a specially prepared mating cap provided. However upon doing so, several important functions are effected and/or initiated. Specifically, the received saliva sample is stabilized and preserved as an additive is introduced to the collected saliva in the sealed container. Further, a lysing reagent acts on cells contained within the saliva to break-down those cells and release component materials therein. Finally, in a purification step, nucleic acid, DNA, or similar genetic material, is separated from other cell matter solution components as DNA is bound to specifically provided DNA binding molecules arranged and provided on a transfer vehicle.

Thus without direct knowledge nor conscious efforts, a user of these systems initiates several important chemical processing steps that are precursors of advanced genetics testing. Time consuming process steps initiated by users of the devices at the time of sample collection are executed and performed thruought the period while the package is in normal transit via common delivery services.

Users of these systems receive a collection kit generally in response to requests for genetics testing services. A kit may be mailed to a requesting user as these kits are durable and safe with respect to those activities associated with common shipping procedures and durations. That is, the kits is stable in time and solutions contained therein have a long shelf life in comparison to delivery schedules. One useful version of a kit includes a single box which contains therein and holds separate two system elements including a receiving vessel and a sealing cap. These may be held separate within the shipping box in a precut foam element having well shaped voids in complementary shapes and sizes with respect to the sealing cap and the receiving vessel. It is important to hold the cap separate from the receiving vessel prior to use as joining the cap to the receiving vessel triggers and releases stored chemistry and processes. Thus, when shipped to a saliva donor, the cap and receiving vessel are said to be held in a decoupled state. After a user receives the kit, the receiving vessel is removed from the shipping box and filled with saliva in accordance with the included instructions. The user/donor then couples the cap with the receiving vessel via a mechanical interlock system supported by both elements—for example a threaded coupling. The operation of coupling and sealing the cap with the receiving vessel causes a reservoir to be breached and for fluids therein to be released from the reservoir and to flow into the cavity containing the collected saliva and thus to mix therewith. This fluid is specialized fluid which contains several components in support of advanced functionality specific to these kits which are particularly adapted for saliva sample collection and transmission by mail. Specifically, the fluid contains a preservative which holds the saliva stabile and protected from degradation for a time period longer than it takes for the package to be mailed to its destination. In addition, the fluid also contains another component which is specifically provided to operate on certain cells which are contained in the saliva. This fluid component performs lysis cells contained in the saliva to release their nucleic matter. Further, in some versions of these systems the fluid additionally contains a mechanical system for DNA purification. Polystyrene or magnetic beads prepared with DNA binding molecules at their surface operate to capture and hold DNA molecules—the beads being suitable for easy separation later. In alternative versions, a vehicle for purification is not contained within the fluid but rather is integrated with the receiving vessel's interior surface. Details follow.

After a user appropriately fills the receiving vessel with saliva and screws the sealing cap to the vessel thus setting them in a coupled state and releasing fluid contained in the reservoir, the user returns the device to the shipping container in a special void in the foam specifically designed to receive cap and vessel coupled together. The box is resealed and readied for shipping or otherwise conveyed to the shipping service for delivery. The same box in which the device arrives at the users location may be used to return ship the sealed container having the saliva sample therein. Because the system is preferably shipped to the user in a decoupled state and returned to the testing facility in a coupled state, the shipping box and foam insert are arranged to support both these configurations.

Collected saliva is first conditioned by a stabilization fluid which provides a safe medium in which saliva will remain for extended periods without damage to the DNA therein and prevent growth of bacteria and/or fungus. In normal anticipated uses of these systems, saliva must be preserved at room temperatures for extended periods. For example, in some cases a sample may be collected and transmitted via the United States Postal Service across the country. In such cases, saliva must be kept in good condition at room temperature for at least 5 days. To effect this, a stabilization fluid used in conjunction with these systems might include sodium benzoate with citric acid. One alternative preservative fluid might also include ethyl and propyl paraben. Of course, other useful preservatives are additionally possible for use as a stabilization fluid for saliva. The essence of these systems does not depend on any particular preservative, and selections from a great plurality of alternatives is possible without deviating from the teachings and advantages of these systems. A preservative which keeps saliva stable for an extended period with respect to typical shipping times is sufficient for purposes of these systems.

In most preferred versions, it is quite useful to begin certain time consuming processes prior to the arrival of the sample at the test facility. Since shipping takes a considerable amount of time, several days in some cases, advantage is realized when this time is put to use to advance the condition and state of the DNA so that it is in best condition for testing upon its arrival. To bring about conditions, DNA must first be released from the cells which contain it. In a preparation process, cell wall membranes are broken-down or otherwise opened to release cell matter into the solution. Accordingly, these systems that include a fluid reservoir integrated with the cap also include a lysis reagent in the fluid.

Lysis may be achieved via several alternative processes each of which has the same result of releasing DNA from the cell nucleus. One preferred method relies upon use of a lysis reagent characterized as a detergent (e.g. Sodium Dodecyl (lauryl) Sulfate (SDS)) Lysis may be carried out in a salt solution containing detergents to denature proteins or proteases (enzymes digesting proteins) such as Proteinase K, or both. Along with the active lysis reagent, a lysis buffer is sometimes used. In some embodiments, a lysis buffer comprised of tris-HCl, EDTA, EGTA, SDS, deoxycholate, and tritonX or NP-40 may be preferred. In some cases useful lysis buffers may also contain NaCl (150 mM).

While many alternative techniques are available to lyse cells, those which agree with the purpose and function of these systems are of particular interest. For example, the lysis reagent used in these systems must be useable with a long shelf life, that is, it must be stable in time. In some preferred versions, the lysis reagent must be mixed with the preservative agent described above and both must exist in the same solution without interfering with the other. Further the lysis agent must not damage nor interfere with purification media nor chemistry. While the precise chemical composition is not critical, the feature described here are necessary attributes of a lysis reagent suitable for these systems.

After saliva is received in the vessel, stabilized with a preservant and cells therein are lysed, the solution is further treated in a purification process. After lysis reagents operate to release nucleic acids from the cells, purification begins. For successful genetics testing, it is necessary to separate DNA from the other components of the lysate. To effect this, the containers presented herein also contain means for binding DNA to transfer vehicle(s) that facilities purification. DNA released from the cells of the saliva is captured and affixed to the transfer vehicle while the other cell matter and other components of the mixture remain free floating in solution. The process to remove DNA from the solution tends to be significantly time consuming and occurs over periods of at least several hours and sometimes up to several days. There exists a natural cooperation between simultaneous execution of the shipping step while the purification process occurs. This is due to the nature of shipping environments which tend to cause the container to be vibrated and agitated. Slight motions and vibrations actually encourage further the binding of DNA to the transfer vehicle. Thus, by starting the purification process prior to shipping these containers realize additional advantage.

One preferred transfer vehicle of these systems includes polystyrene beads prepared with DNA binding molecules at their surface. When present in solution with DNA, the DNA will bind to the bead and be affixed thereto. It processing steps executed later, the beads with the DNA affixed may be easily separated from the other components of the solution. For purposes of this description, the polystyrene beads with their prepared surface of DNA binding molecules is considered the transfer vehicle. In one version, the polystyrene beads so prepared may be included within the composition of fluid held in the reservoir.

In one important alternative version, a transfer vehicle may be arranged as magnetic beads with similar DNA binding molecules or sites on its surface. In a similar fashion, these beads will capture and hold fixed free floating DNA molecules of the lysate. This further facilitates removal of DNA from the solution in a purification step. Magnetic beads are particularly easy to separate from the other solution components due to magnetic force attractions which do not act on components of the solution other than the magnetic beads. When the container arrives after several days in transit, a magnetic force is applied to the solution to draw the beads (and attached DNA) away from the solution in a washing step. During transit, DNA continuously binds to the magnetic beads over the course of an extended period. Accordingly, another important transfer vehicle in these systems are magnetic beads prepared with DNA binding molecules at their surface. These magnetic beads may also be included as part of the fluid solution contained in the reservoir that gets released upon coupling of the sealing cap and the receiving vessel.

Some versions of these systems do not include a purification transfer vehicle as part of the fluid contained in the reservoir. Rather, a transfer vehicle may be incorporated as part of the receiving vessel itself. DNA binding molecules may be prepared and affixed to an inside surface of the receiving vessel. This is a particularly attractive alternative in cases where it is desirable to have a very large surface area for expedited purification. Beads included in a fluid have limited surface area and purification rates are slower. Where a large surface area can include many more DNA binding sites, the purification may occur at a higher rate and at a higher efficiency. Thus, where some high performance devices are required, a transfer vehicle may be provided as DNA binding molecules attached to the inside surface of the receiving vessel.

Accordingly, this invention also includes methods of collecting biological samples. Methods of this invention are characterized as having the steps: receiving a sample, adding to the sample either of the following from the group: preservative and/or stabilization agents; lysis reagent(s); and purification systems, and finally conveying the sample by delivery services for example the public mails or private express delivery.

When a sample is put into an appropriately designed vial system or receiving vessel and that vial is joined by its complementary and cooperating cap, fluid is released into the sample while at the same time a seal between the cap and the vessel form a liquid tight enclosure. Further, fluid released from the reservoir is mixed with and interacts with the collected sample. Thus, in versions which include a purification system, an important step of the method includes bringing into proximity the purification transfer vehicle and the sample collected (saliva). This may come about by either mixing a fluid containing polystyrene beads with the sample, or merely allowing the sample to contact prepared interior walls of the receiving vessel—the walls having thereon DNA binding molecules. In an alternative version of a purification system, the fluid contains beads or particles of a magnetic nature those magnetic beads also having DNA binding particles thereon.

In all method versions, the collected sample is treaded with fluid from the reservoir and sealed in the container for shipping. The container may then be placed into a specially prepared shipping box for return shipping to a test facility. A user may use the same box as received by applying a return shipping label of an original label and depositing the package with a shipping service. A core element inside the box supports return shipping of the coupled cap and vessel.

While it is a most convenient illustration to use DNA in saliva for examples developed thruought this disclosure, the reader will appreciate that these systems are not limited to either saliva or DNA. Any biological matter such as blood, sweat, cheek swaps, et cetera may cooperate with various versions of the same concept. Indeed, from the biological material DNA or other nucleic acids are not the only function target molecule. Some version of these systems are designed to target biological molecules which are not nucleic acid. These might include hormones, proteins, lipids, peptides, among others. A purification transfer vehicle of these systems may be designed to capture and isolate any of these target molecules while leaving all other non selected matter in the solution to facility isolation of the target molecule in a washing step. Indeed some purification transfer vehicles of these systems also might be designed to target a combination of of those target molecules. This is realized in the design of the purification agent. Whether the transfer vehicle is arranged as free floating beads or the inner walls of the receiving vessel, in both cases a capture or binding molecule is designed to firstly be affixed to a surface of the transfer vehicle and secondly to attach and bind to the design target molecule which is suspended in a solution. Accordingly, these systems shall be considered inclusive of target molecules which are not DNA but nevertheless of interest in testing applications. Examples set forth herein are primarily directed to DNA related cases and saliva as these set forth a most clear illustration. It is not intended except where explicitly set forth in the claims that the invention be limited to either saliva or DNA.

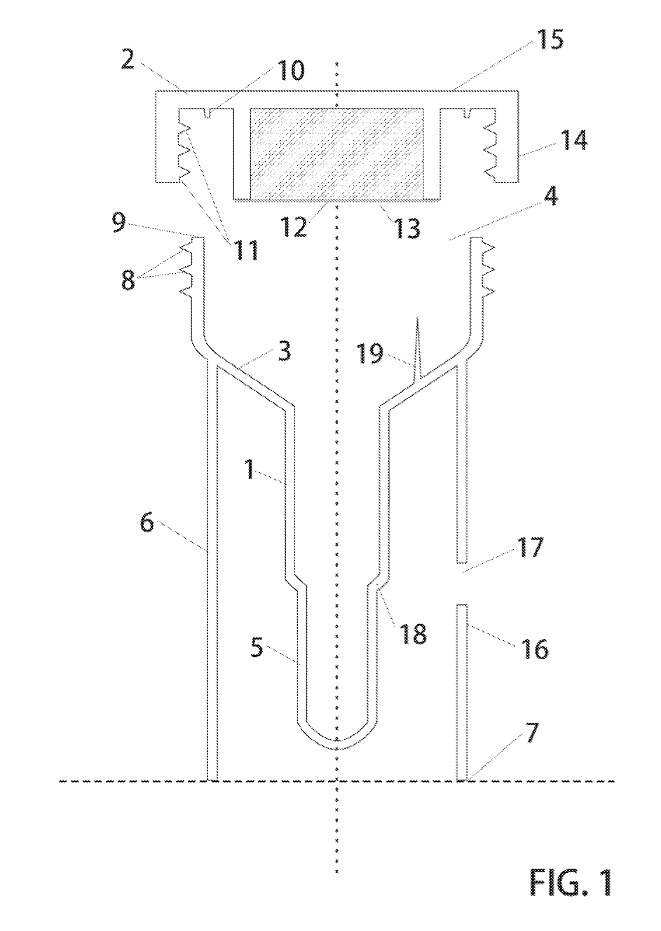

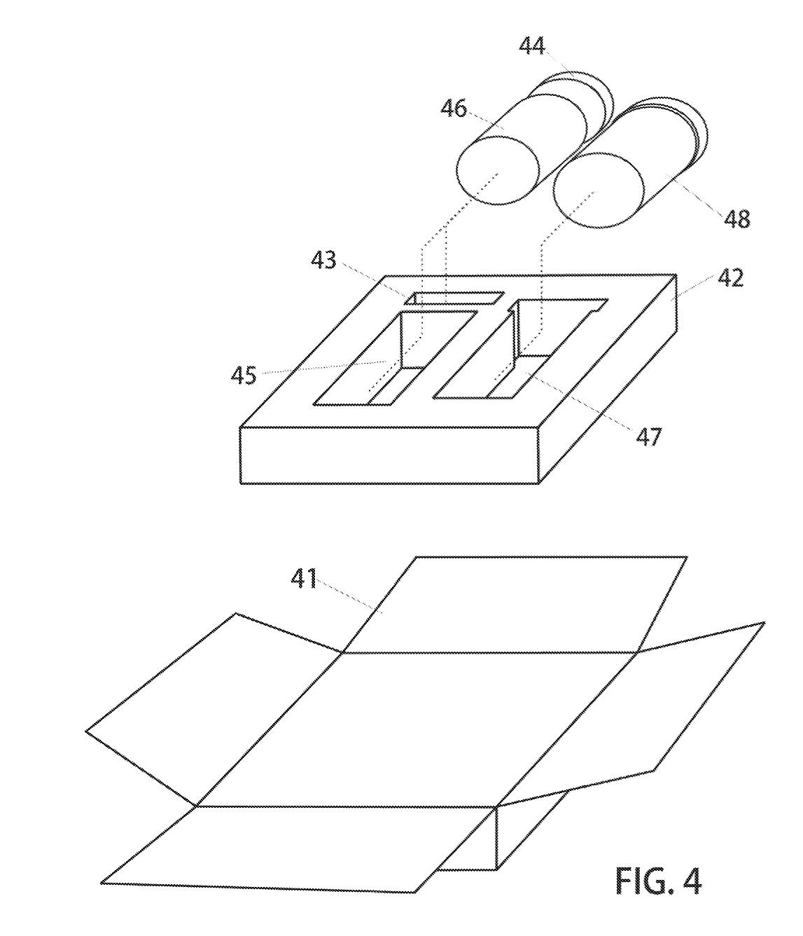

By way of illustrative examples, one will gain a further detailed understanding of these systems. With attention to the appended drawing FIGS. 1-4 and particularly reference numerals therein, one gains a firm and complete understanding of system apparatus of these teachings.

Specifically, FIG. 1 illustrates in cross-sectional view of two primary elements in a spatially proximate relationship on a symmetry axis. Receiving vessel 1 is a cooperating complement part to a sealing cap 2. These are arranged in the diagram such that one aspect (a mechanical interlock system; e.g. thread set coupling) of each of their geometries cooperates with the other and includes means for tight coupling. The receiving vessel element includes an integrated funnel portion 3 with an entrance aperture 4 and elongated cylindrical tubular portion 5 which lies coaxially with respect to a second cylindrical structure a rigid body 6 including terminus annulus 7 lying in a plane forming a ‘foot’ or ‘base’. At the top of the receiving vessel element at an outside peripheral edge, a thread set system 8 is formed as a part of a mechanical interlock or coupling means between the receiving vessel element and the cap element. This coupling may further include a well-designed seating surface 9 to cooperate with an annular flange 10 integrated with the sealing cap to form a liquid tight seal. While preferred versions include those where the flange is molded integrally with the cap, other version include those where pressure fit receiving space holds an ‘O’-ring to the sealing cap interior. When a sealing cap arranged accordingly is coupled with the receiving vessel, the cap forms a more durable liquid-tight seal with the receiving vessel at the ‘O’-ring and corresponding and cooperating seat. A complementary and cooperating thread set 11 is formed on an inside cylindrical surface of the cap. When a cap so described and receiving vessel similarly so, are brought together and rotated about an axis in opposing directions, a liquid tight seal is formed between them as the threads are arranged such that the annular flange is pressure fitted to the seating surface of the receiving vessel. In addition to forming a liquid tight seal between the cap element and the receiving vessel element, the act of bringing these two elements together via this threaded coupling invokes another important function. A liquid tight reservoir 12 contains therein a special formula fluid solution. This fluid solution may include several primary components amongst which include a stabilization compound, lysis reagent, and optionally components of a purification system. These components operate to stabilize and preserve, lyse, and purify matter of a biological sample such as saliva. The reservoir is comprised in part of one functional surface which may be pierced or otherwise compromised such as a thin-film membrane or foil 13. When the sealing cap is screwed onto the receiving vessel it advances in an axial direction towards the receiving vessel. A carefully positioned knife 19 integrated with the receiving vessel is provided to pierce the membrane and cause the liquid contained therein to leave via gravity. Accordingly, bringing the cap into relation with the receiving vessel assures release of the formula from the reservoir and further assures it mixes with the collected and contained saliva.

While this illustrates an excellent example, it is not difficult for packaging engineers to arrange for the release of fluid in response to a cap being secured to a receiving vessel. Alternatives will be considered merely engineering advances as the invention here contemplates a great plurality of arrangements of reservoirs which yield their contents as a sealing cap is joined with its mating part. As such, the invention does not rely on any precise positioning of the knife as shown in the illustrative example, but rather upon the notion that merely joining two component parts sets into motion a stabilization, lysis and purification process with respect to DNA in saliva.

The cap element may additionally include a knurled outer surface 14 and label receiving surface 15. The knurled outer surface promotes ease-of-use by permitting good ergonomic cooperation with the human fingers whereby the cap may be tightly coupled to the receiving vessel despite some resisting pressure due in-part to friction between the thin film membrane and knife and further the annular flange and its seat. The label receiving surface is adopted to accept by way of adhesives a label so that these containers might more easily be marked with identifying information. It is generally convenient to associate the contents of a used container with a particular donor and sometimes this is done by way of a label affixed to the cap which does not easily separate therefrom and remains quite accessible for every person in the processing chain.

Finally, the receiving vessel element may additionally have integrated therewith indicia 16 in support to promote ease-of-use. A “fill-line” mark can be included on the tubular cylindrical portion of the receiving vessel or other outside surface of the body. Other indicia may be also provided to similarly promote proper use.

In some preferred versions, a slot may be provided in the body to effect a viewing window 17 by which a user may view more easily the tubular portion which contains collected sample matter (saliva). More precisely, to view a ridge 18 formed into the tubular portion which operates as a fill-line or fill-limit.

With a clear understanding of each of these systems integrated with either of the two primary elements, the receiving vessel element or the cap element, a more detailed description of each follows as various versions are further described.

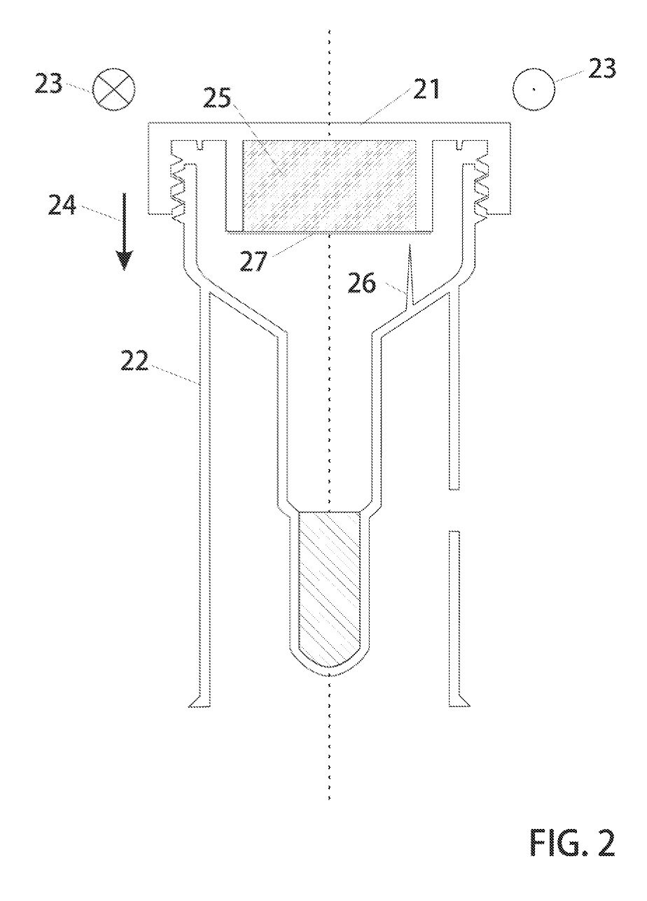

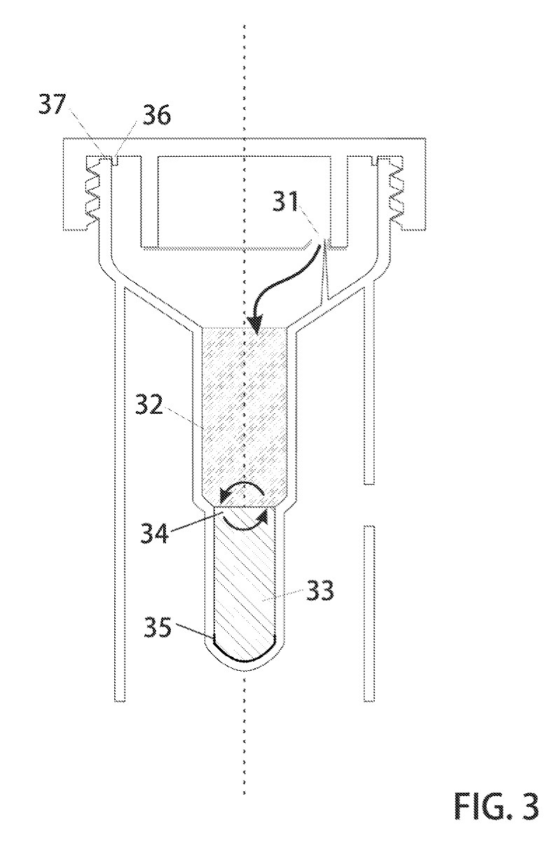

FIGS. 2 and 3 further illustrate important aspects of these systems including the coupling between a sealing cap 21 and receiving vessel 22. By way of counter-rotation (indicators 23) about a common axis, a cap is screwed onto the receiving vessel to couple them together and form a liquid tight seal therebetween. As the cap is turned, the thread set causes the cap to advance towards (indicated by 24) the receiving vessel along the axis. As the reservoir 25 containing a fluid solution of several components therein advances to the knife 26 and conies into contact therewith, the thin-film 27 is pierced thus releasing the contents of the reservoir into the cavity in which the saliva is held. In most preferred versions, the contents of the reservoir may include a preservative fluid, a lysis reagent, and a component of purification means. In some alternative versions, components of the purification means are omitted from the fluid and incorporated instead with the interior surface of the tube of the receiving vessel. In those cases, fluid confined to the reservoir and subject to release via the piercable seal only include a preserving solution and a lysis reagent.

FIG. 3 illustrates further the release of the reservoir contents into the sealed cavity in which saliva is contained and the simultaneous formation of a seal between the cap and receiving vessel. The plastic knife integrated with the receiving vessel for example in a molding process, pierces 31 the thin membrane of the sealing cap reservoir to release the fluid 32 containing at least preservative and lysis reagent and sometimes a purification component whereby the fluid solution passes into the receiving vessel to mix 33 with collected saliva 34.

In some special versions in which the reservoir is prepared with only a preservative and a lysis reagent but not purification means, the inside surface 35 of the tube may be prepared with a DNA purification system. Specifically, the surface of the tube may be prepared to include DNA binding molecules which cause free floating DNA in the lysate to be captured, affixed and held to the surface. This promotes an easy wash step which may be executed in the laboratory after the container system is received from the conveying donor. As the container spends a significant amount of time in transit, it is quite advantageous to simultaneously perform the DNA separation step (this step similarly requires a considerable amount of time). In some best versions, these systems benefit from the very large surface area of the interior surface of the receiving vessel. When that surface is covered with DNA binding molecules, the efficiency of separating DNA from the solution is quite good. Accordingly, a container described herein can be received from the shipping service with the DNA largely separated from the solution via this transfer vehicle incorporated with the interior surface of the receiving vessel. As such, a considerable amount of time is saved as the container is received in a condition to immediately begin the washing processes.

As an unskilled user screws the cap to the receiving vessel to release the fluid solution of the reservoir, the cap forms a liquid tight secure seal with the receiving vessel. The cap is further advanced to its seat such that a liquid-tight seal is formed between the annular flange of the sealing cap 36 and an inside surface 37 of the receiving vessel which forms a cooperating seat. A pressure between these two elements assures liquid is securely contained within the receiving vessel for a durable and long-term storage. While good package engineers can devise many alternative means of forming a liquid tight seal, it will be recognized that alternative seals will not deviate from the essence of these teachings with regard to pre-processing of saliva samples in containers subject to long transit periods.

One important auxiliary aspect of these systems relates to use of the apparatus during a collection step. As the quantity of saliva a required tends to be more than what might be obtained in a single ‘spit’ action, a donor is required to a repeatedly spit into the funnel aperture. For this reason, it is desirable that the apparatus can be placed on a flat surface to rest between spits. To provide for this, a outer tube fashioned as a rigid body is shaped with a base for ‘foot’ portion to assure the vessel is held upright when resting on a table. While the base may include an opening at its bottom, at least an annular ring which lies substantially in a plane provides a resting surface for these devices. Some versions may include a flared lip on the body to increase the surface area of the annulus for improved stability.

The rigid body is sometimes transparent or translucent for an attractive ‘clean’ feel and may be formed of molded plastic. In addition, its outside surface may be scored or scuffed to provide for an improved grip and handling. As some molded plastics are left quite smooth after formation, it is sometimes desirable for these apparatus to prepare the outside surface of the body has a ‘gripping’ surface economically suitable for being handled easily by human fingertips.

A reservoir is preferably integrated within an interior cavity portion of the cap element. The reservoir may be formed integrally with the cap in a plastic molding step. It is preferably cylindrical in shape and approximately 1 cm deep. The reservoir may be filled with a preserving fluid by automated machinery, and thereafter the reservoir may be sealed to contain and protect the fluid until it is required for use. In anticipated systems, a reservoir may contain a specially prepared bonding surface to which a thin-film or foil may be affixed for example by adhesives or plastic weld. A thin-film or foil is used to complete the reservoir cell. As these support functionality related to release of the fluid from the reservoir at the appropriate time. Namely, when a cap element is coupled to the receiving vessel element, it is desirable to automatically have the fluid mixed with the saliva. Accordingly, without taking any extra steps, measuring, pouring, calculation, et cetera, an unskilled user preserves the saliva sample merely by screwing the cap to the receiving vessel together.

In one preferred version, the fluid solution contained in the reservoir is colored with a dye agent. This yields a mechanism by which a user can easily determine that the foil seal was appropriately compromised and that fluid previously contained in the reservoir has been fully released therefrom and has further mixed with the saliva sample. In systems where a dye is not used, it has been observed that failure due to malfunction of the foil piercing system went undetected in the sample subsequently was spoiled.

Another important aspect of these saliva collection systems includes a special dual-mode shipping container. As these systems are specifically configured for use directly by consumers, for example at a consumer's private home, it is advantageous to provide for two-way shipping in a container suitable for same. A box 41 may have snugly inserted therein a core element 42, for example a core element made of shaped foam material. The core, element has a periphery, for example an exterior surface which is similar in shape and size with respect to the shipping box to permit it to snugly fit therein. The shipping container including this foam core element by way of cut-out cavities therein supports two discrete shipping modes. When a saliva sample collection device in accordance with this invention is shipped to a consumer, it is necessary that the cap and receiving vessel be held separate to protect the contents of the fluid reservoir against being released. Accordingly in this first shipping mode a first cut-out cavity 43 is provided to accommodate the cap 44 containing the reservoir therein, while a second cut-out cavity 45 is provided to accommodate the receiving vessel 46. Once inserted accordingly, the cap and receiving vessel are held isolated spatially thereby maintaining the integrity of the reservoir. After use, and once a cap is tightly coupled with a receiving vessel and fluid has been released from its containment in the reservoir, the combined cap and receiving vessel 47 may be inserted into another cut-out cavity 48 provided specifically for the combination such that the system including the sample mixed with preserving fluid may be return shipped to the laboratory. In this manner, the shipping box is particularly arranged to cooperate with the objective of releasing the fluid solution only after use. Additionally, both shipping modes are supported by a single container and no waste material remains further improving the efficiency of the kit. A kit might additionally include an instruction booklet 49 which illustrates proper use of the system. Additionally in some kits, a return mailing label which can be easily applied over a first ‘ship to’ label and also is included a an element of these kits.

In accordance with each of preferred embodiments of the invention, saliva collection, preservation, lysing, purification and conveyance systems are provided. It will be appreciated that each of the embodiments described include an apparatus and that the apparatus of one preferred embodiment may be slightly different than the apparatus of another embodiment. Accordingly, limitations read in one example should not be carried forward and implicitly assumed to be part of an alternative example.

What is claimed is:

1. A sample collection apparatus for collection and conveyance of biological matter comprising:
a receiving vessel and cooperating sealing cap, each comprising a complementary mechanical interlock system operable for forming a liquid tight seal therebetween when in a coupled state.

2. The collection apparatus of claim 1, wherein either of said sealing cap or receiving vessel further comprises a reservoir of fluid solution, the fluid being releasable from said reservoir upon coupling between said sealing cap and receiving vessel.

3. The collection apparatus of claim 2, said reservoir having therein a fluid solution characterized as comprising a preserving fluid, wherein said preserving fluid inhibits growth of bacteria and/or fungus.

4. The collection apparatus of claim 3, wherein said preserving fluid is characterized as a fluid that inhibits degradation of genetic matter.

5. The collection apparatus of claim 3, wherein said fluid solution further comprises a lysis reagent.

6. The collection apparatus of claim 3, wherein said fluid solution further comprises a purification agent.

7. The collection apparatus of claim 5, wherein said fluid solution further comprises a purification agent.

8. The collection apparatus of claim 7, wherein said purification agent is further characterized as comprising beads having affixed at an external surface biological target binding molecules.

9. The collection apparatus of claim 8, wherein said beads are characterized as magnetic beads or magnetic particles.

10. The collection apparatus of claim 8, wherein said beads are characterized as plastic or polystyrene beads.

11. The collection apparatus of claim 3, further comprising a purification system arranged as an interior surface of the receiving vessel having biological target binding molecules affixed thereto.

12. The collection apparatus of claim 5, wherein mechanical interlock is a screw thread set integrated with each of said sealing cap and receiving vessel.

13. The collection apparatus of claim 5, wherein said reservoir has thin pierceable-membrane which forms a sealed enclosed cavity which contains fluid therein.

14. The collection apparatus of claim 13, wherein knife is integrated with the receiving vessel interior walls.

15. The collection apparatus of claim 14, wherein said reservoir is well aligned with knife in view of rotation axis.

16. The collection apparatus of claim 2, said apparatus further comprising a purification system comprising a transfer vehicle having thereon biological target binding molecules.

17. A method of sample collection comprising the steps of:
receiving a sample of biological matter into a receiving cavity;
adding to said received sample a preservative;
adding to said received sample a lysis reagent; and
conveying said sample via shipping services.

18. The method of sample collection of claim 17, further comprising the step of bringing into proximity of said received sample, a purification system.

19. The method of sample collection of claim 18, wherein said purification system comprises magnetic beads having thereon biological target binding molecules.

20. The method of sample collection of claim 18, wherein said purification system comprises polystyrene beads having thereon biological target binding molecules.

21. The method of sample collection of claim 18, wherein said purification system comprises an internal surface of a receiving vessel having thereon biological target binding molecules.

22. A kit for sample collection and conveyance by mail or delivery services, said kits comprising:
a receiving vessel;
a sealing cap;
a shipping box; and a
core element,
said core element having a periphery which cooperates with the interior surface of the shipping box to snugly fit therein, the core element further having cut-out regions complementary in shape and size with respect to said receiving vessel and sealing cap whereby they may be securely held therein, the core element further comprising a second cut-out region complementary in shape and size with respect to a coupled receiving vessel and sealing cap.

23. The sample collection kit of claim 22, said sealing cap further comprising a reservoir having a piercable thin membrane seal and fluid solution contained therein, said fluid solution comprising a preservative and a lysis reagent.

24. The sample collection kit of claim 23, further comprising a purification system, said purification system being characterized as a transfer vehicle having thereon its surfaces biological target binding molecules.

25. The sample collection kit of claim 24, further comprising a two-way shipping label system and an instruction set.

26. The sample collection kit of claim 23, wherein said core element is further characterized as a shaped paperboard having receiving voids therein whereby the sealing cap and receiving vessel may be held in either a coupled or decoupled state.

27. The sample collection kit of claim 22, wherein said receiving cap is comprised of a reservoir of fluid solution, said reservoir having a release mechanism when initiated releases the reservoir contents into said receiving vessel.

28. The sample collection kit of claim 27, wherein said receiving cavity has integrated therewith a protrusion operably located to pierce said membrane as a sealing cap is brought into relation with said sealing vessel.

29. The sample collection kit of claim 28, wherein said fluid solution comprises a preservative solution and a lysis reagent.

30. The sample collection kit of claim 29, wherein said fluid solution comprises a purification transfer vehicle.
